(19) United States
(12) Patent Application Publication
(10) Pub. No.: US 2004/0002722 A1
(43) Pub. Date: Jan. 1, 2004

(54) Ultrasonic microkeratome

(76) Inventor: Stephen G. Slade, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 10/384,322

(22) Filed: Mar. 7, 2003

Related U.S. Application Data
(60) Provisional application No. 60/362,305, filed on Mar. 7, 2002.

(51) Int. Cl. A61B 17/32

(52) U.S. Cl. 606/166; 606/169

(57) ABSTRACT

The invention relates to medical instruments and methods for performing eye surgery to correct focusing deficiencies of the cornea. More particularly, the present invention relates to mechanical instruments known as microkeratomes, and related surgical methods for performing lamellar keratotomies and refractive surgery. The device is designed to create a flap of epithelium, or stroma and epithelial flap as is done now with LASIK. This will enable a new technique, ELF, or Epithelial Laser Flap, which will combine the advantages of LASIK and PRK, an older, but easier technique.

### CROSS-REFERENCE TO RELATED APPLICATIONS

This application claims priority to U.S. Provisional Application 60/362,305, filed Mar. 7, 2002.

### STATEMENT REGARDING FEDERALLY SPONSORED RESEARCH OR DEVELOPMENT

Not Applicable.

### REFERENCE TO A MICROFICHE APPENDIX

Not Applicable.

### BACKGROUND OF THE INVENTION

1. Field of the Invention

The present invention relates to medical instruments and methods for performing eye surgery to correct irregularities of the cornea. More particularly, the present invention relates to mechanical instruments known as microkeratomes, and related surgical methods for performing lamellar keratotomies and refractive surgery.

2. General Background of the Invention

In recent years, as Refractive Surgery has developed, a number of surgical techniques have become available to surgically treat near sightedness, farsightedness and astigmatism. Of these surgical techniques, laser in situ keratomileusis (LASIK) has evolved into one of the most promising members in the family of lamellar refractive surgeries. LASIK has recently gained popularity for the correction of myopia because of the reduced risk of post-operative corneal haze, rapid visual rehabilitation, pain-free post-operative course, and decreased need for post-operative medications.

LASIK surgery consists of cutting a flap of cornea stroma and epithelium, lifting the flap and reshaping the exposed bed with an excimer laser. The flap is then repositioned and seals itself down. A microkeratome is used to fashion the flap. The microkeratome is generally a blade carrying device which functions like a carpenter's plane or surgical dermatome, that may be manually pushed or mechanically driven in a cutting path across a suction ring simultaneous with the motorized movement of the cutting element, which movement is transverse to the direction of the cutting path.

The microkeratome includes the suction or guide ring, which is fixed to an ocular globe, or eyeball, with the aid of a partial vacuum applied through the ring. The suction ring immobilizes the ocular globe, maintains the tension of the globe, and regulates the diameter of the corneal resection. A portion of the microkeratome called a cutting head is supported within a channel in the suction ring for guided linear movement of the microkeratome across the suction ring by the surgeon.

Jose Ignacio Barraquer at his clinic in Bogotá, Columbia began fifty years ago to develop the concept of lamellar refractive corneal surgery. He conceptualized that by removing corneal stroma tissue that the tear film-anterior cornea interface would be flattened and alter the refractive power of the eye. He reported his first results in 1949. The surgical term for the techniques that Dr. Barraquer developed was keratomileusis, which is derived from the Greek roots keras (horn-like—cornea) and smileusis (carving).

Dr. Barraquer's initial technique consisted of performing a freehand lamellar dissection with a Paufique Knife or corneal dissector to create a 300-micron corneal lamellar disc. He then attempted the refractive cut by removing stroma from either the bed (keratomileusis in situ) or from the posterior aspect of the corneal lamellar disc. Due to poor success with removing stroma from the bed either manually with the Paufique knife or a manual keratome he abandoned the in situ approach and refined the lamellar dissection and precise carving of the corneal lamellar disc.

Krwawicz and Pureskin, working independently in the 1960s, developed techniques to remove central lamellae of stroma, but it was Barraquer's persistence that led to several cornerstones for modern keratomileusis principles and instruments. He came to understand the interrelationship between suction ring induced intraocular pressure and corneal disc diameter to the thickness of the resected disc of tissue. With Dr. Barraquer's groundbreaking work and subsequent research into sculpting the corneal disc with a cryolathe he laid the basis for modern myopic keratomileusis, hyperopic keratomileusis, keratophakia and LASIK.

Several drawbacks, however, were inherent to Dr. Barraquer's initial techniques which included the complex nature of the procedure and instrumentation, low margin for error, and steep surgeon learning curve. Additionally, since the keratectomies were done by free hand, the resection depended on a steady rate of passage, adequate suction and good cent ration. If a good keratectomy was not achieved, interface scarring, an irregularly thin corneal disc and ultimately irregular astigmatism could be experienced.

The critical step for achieving the best results in myopic keratomileusis in situ is the controlled pass of the microkeratome. Luis Ruiz in the late 1980's developed a foot operated automated geared microkeratome This microkeratome provided a more consistent cut due to controlled speed, and the keratectomy displayed a very smooth surface. Subsequently new and better keratomes such as the Hansatome have added precision and safety to lamellar surgery.

Researchers felt the excimer laser with its ability to remove fractions of a micron with each pulse would allow surgeons to precisely correct both spherical and astigmatic errors better than the cryolathe or recutting with the keratome. Peyman, in 1989, reported the first animal study in which a laser was used to remove corneal stroma from a lamellar bed. Lucio Buratto presented the first work with Excimer Laser Keratomileusis. He elected to use the excimer laser to ablate the backside of the corneal cap (disc) that he achieved with the first pass with the Barraquer-Krumriech-Swinger microkeratome. Pallikaris performed excimer laser ablation on the stroma bed in rabbits and also compared excimer laser keratomileusis in situ to photorefractive keratectomy histopathologically. Slade soon thereafter ablated the stroma bed after making a nasally based corneal hinged-flap with the automatic geared microkeratome developed by Ruiz. In short, this progression to LASIK helped to achieve extreme precision of tissue removal with the excimer laser without exposing the operated area to the healing processes of the eye (lamellar technique) along with providing the patient a more comfortable recuperative process.

LASIK is the marriage of lamellar corneal techniques that have been under development for approximately the past 50 years and the extreme precision of the argon-fluoride excimer laser at wavelength 193 nanometers that has brought about this excitement. LASIK has largely replaced an older technique, Photorefractive keratoplasty (PRK). PRK surgery consists of the top layer of the cornea, the epithelium, being scraped away and discarded. The laser is then used to shape the exposed surface. After the laser application, the epithelium must regrow which can be a painful process with reduced vision for several days.

LASIK has several advantages to PRK, all related to the flap of tissue that is created. Clinicians worldwide have reported on the limited efficacy of photorefractive keratectomy with the excimer laser for patients with greater than −6.00 diopters and even more so with patients with greater than −10.00 diopters of myopia. Further, the development of postoperative scarring in the central cornea after excimer laser surface ablations has resulted in regression of effect, significant disturbing visual complaints and lines of lost best-corrected vision.

Several reports have been made on the wound healing response after photorefractive keratectomy and its pharmacological modulation in addition to studies on retreatments due to undercorrection. Additionally, the postoperative discomfort and the relatively long postoperative recuperative period after surface ablation is currently an inescapable reality for both the patient and the eye care professional. For these core reasons and likely several others, refractive surgeons were left wanting for a better technique and thus have continued research with lamellar corneal surgery and excimer lasers in hope of a better surgery from both a patient satisfaction standpoint and improved refractive predictability.

While LASIK offers several of advantages over PRK, the creation of the corneal flap has been associated with a number of intra-operative and post-operative complications. Because the ablation is started beneath the corneal flap, 160 microns deep, there is a limited amount of tissue left in the typical 540-micron cornea. Research has shown, and the FDA pronounced, that a residual bed of 250 microns should be left in all cases. Thus some patients with high myopia or thin corneas are left without a treatment option.

Second, cutting the flap in the cornea alters the biomechanical properties of the cornea and may induce aberrations or irregularities in the surface of the cornea. Third, the incidence of complications involving the flap, while small, is significant. The most common post-operative complications associated with the corneal flap include flap striae and epithelial ingrowth. Thus some surgeons have taken another look at PRK.

Some surgeons have adopted a new form of PRK, LASEK, or laser epithelial keratoplasty, as a way to avoid the problems in LASIK. In LASEK surgery a flap of epithelium is fashioned by hand held instruments and turned back The cornea is then reshaped, and the epithelium is repositioned. The recovery is slower than LASIK, as the flap usually sloughs and has to regrow. Also, to help remove the epithelium drugs such as alcohol must be used in LASEK to loosen the epithelium, damaging the epithelium and slowing recovery. As a result, patients have more discomfort and a slower return of good vision. The technique also is surgically tedious and difficult.

We propose an automated device to cut an epithelial flap quickly and reproducibly with a minimum of manipulation to the cornea. This technique, Epithelial Laser Flap, (ELF) would offer a quick recovery and surgical time of LASIK but with the safety, tissue preservation and reduced complications rate of PRK.

### SUMMARY OF THE INVENTION

The present invention is designed to satisfy a need in lamellar surgery and is directed towards a new and improved automatic surgical device known as an epithelial layer cutter (ELC). The present invention is designed to cut and lift a thin layer of epithelium on a patient's eye and to create a hinged flap of epithelial tissue. Morever, the present invention is designed to cut into the cornea to create a hinged flap of corneal tissue, for example under the Bowman's membrane or into the cornea stroma.

The present invention includes a means for retaining and positioning the eye on which surgery is to be performed, a cutting head assembly including a cutting element positioned therein for lifting the epithelium of the eye, and a coupling member for detachably coupling the retaining and positioning means and cutting head assembly while permitting movement of the cutting head assembly relative to the retaining and positioning means along a generally arcuate or longitudinal path.

### BRIEF DESCRIPTION OF THE DRAWINGS

A better understanding of the invention can be obtained from the detailed description of exemplary embodiments set forth below, when considered in conjunction with the appended drawings, in which:

FIG. 1 is a schematic illustration of the cornea;

FIG. 2 is an illustration of flap dimensions;

FIG. 3 is an illustration of a flap being cut;

FIG. 4 is an illustration of a flap being cut;

FIG. 5 is an illustration of a flap being lifted after cut;

FIG. 6 is an illustration of various suction ring views;

FIG. 7 is an illustration of various hand piece views;

FIG. 8 is an illustration of views of embodiments of the present invention; and

FIG. 9 is an illustration of an ultrasonic control console.

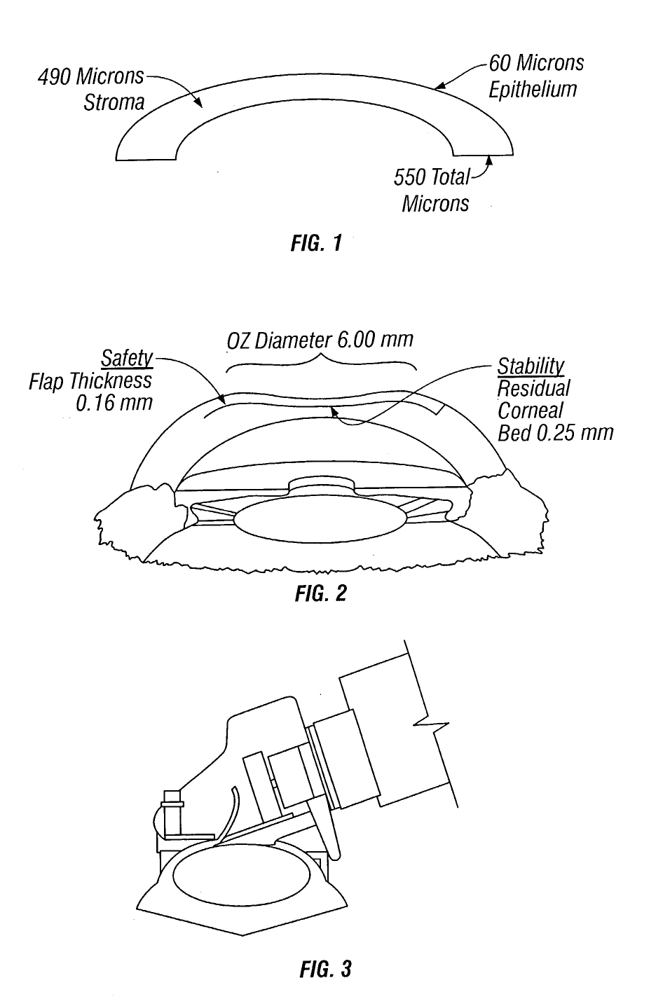

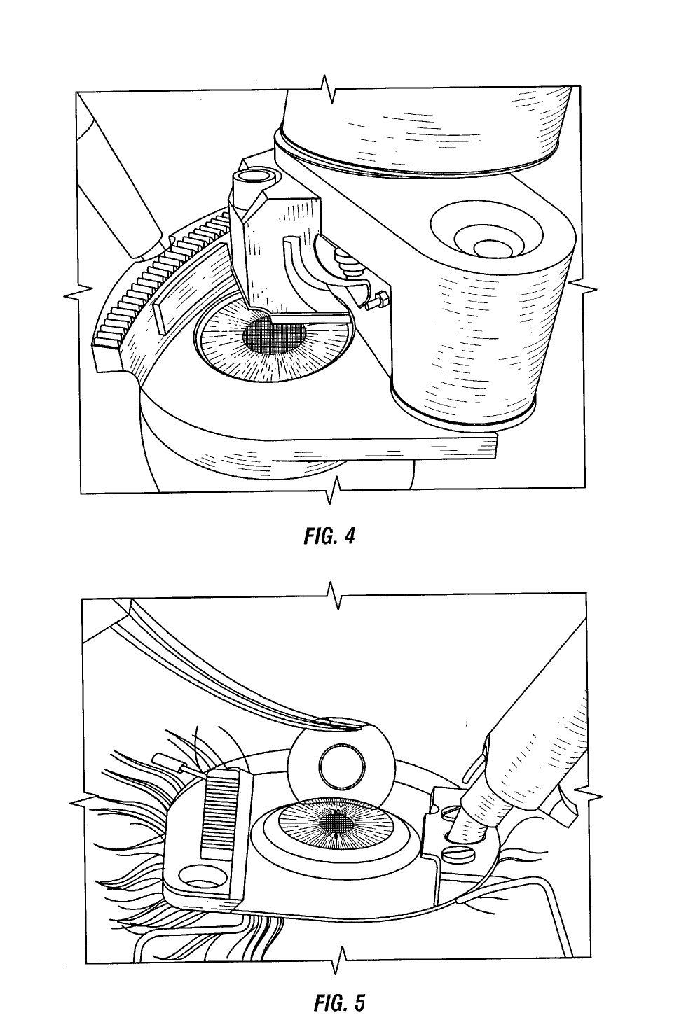

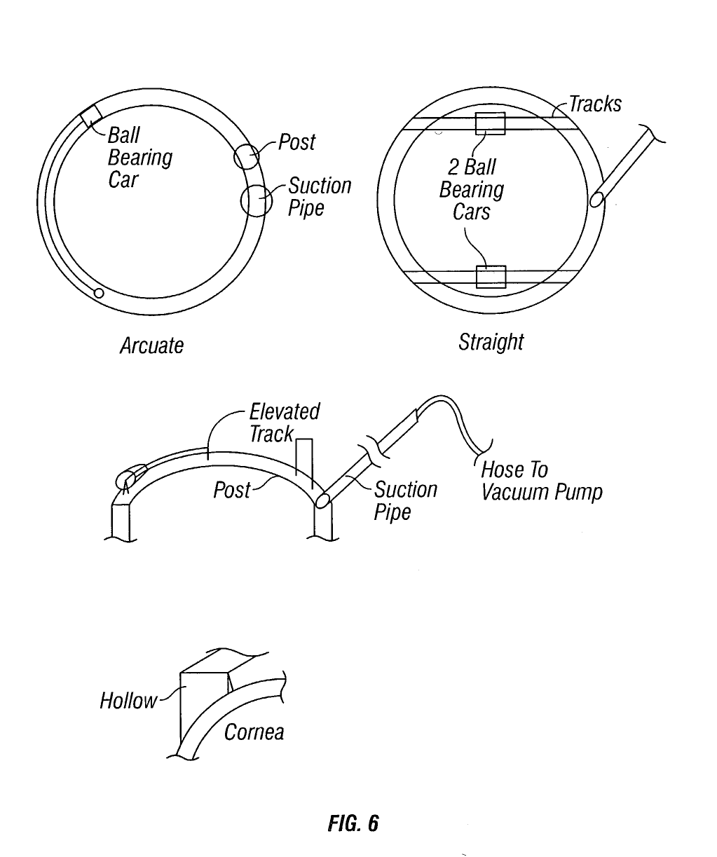

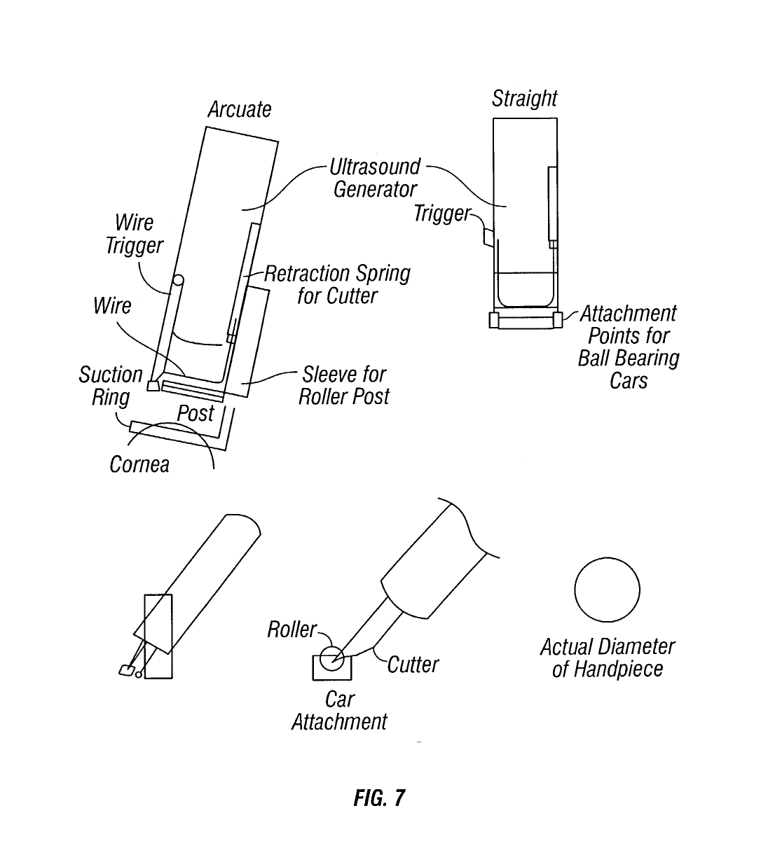

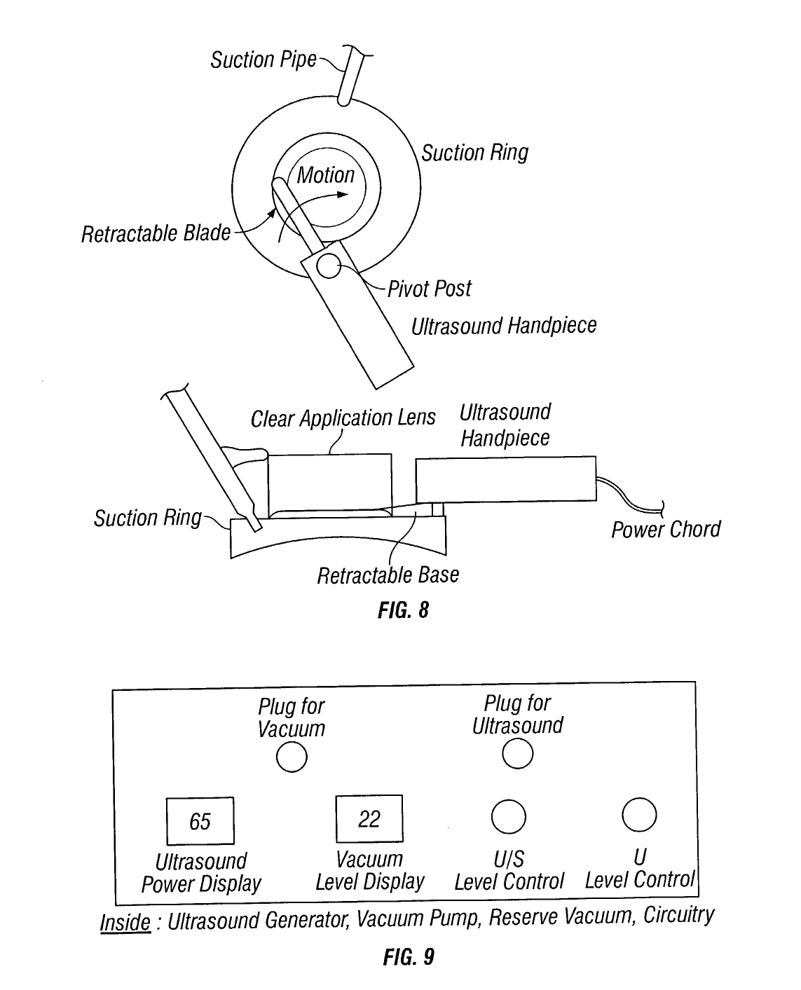

### DETAILED DESCRIPTION OF EXEMPLARY EMBODIMENTS

The eye works on a principle very similar to that of a camera. The iris or colored portion of the eye about the pupil, functions like a shutter to regulate the amount of light admitted to the interior of the eye.

The cornea or clear window of the eye, and the lens, which is located behind the pupil, serve to focus the light rays from an object being viewed onto the retina at the back of the eye. The cornea is composed of five layers; first the epithelium that is five cells thick and is usually around 60 microns thick. A thin membrane called Bowman's membrane underlies the epithelium. The mass of the cornea is called the stroma, which is about 480 microns thick. The fourth layer is another, stronger but very thin membrane called Descemet's. The final layer is the endothelium, which is only one cell thick. Bowman's, Descemet's and the endothelium do not contribute significantly to the total cornea thickness. The total thickness of the cornea averages around 540 microns. Once the cornea and lens focus the rays of light on the retina, the retina then transmits the image of the object viewed to the brain via the optic nerve. Normally, these light rays will be focused exactly on the retina, which permits the distant object to be seen distinctly and clearly. Deviations from the normal shape of the corneal surface, however, produce errors of refraction in the visual process so that the eye becomes unable to focus the image of the distant object on the retina. Hyperopia or “farsightedness” is an error of refraction in which the light rays from a distant object are brought to focus at a point behind the retina, as indicated by the solid lines. Myopia or “nearsightedness” is an error of refraction in which the light rays from a distant object are brought to focus in front of the retina, as indicated by the solid lines, such that when the rays reach the retina, they become divergent, forming a circle of diffusion and consequently, a blurred image.

In one embodiment, the retaining and positioning means comprise a suction ring having means for temporary attachment to a portion of the eye surrounding the cornea to be cut, and which expose and present the cornea for cutting. The suction ring or other retaining and positioning means includes a guide means thereon, preferably disposed on an upper surface thereof and extending in a generally arcuate or straight path. The suction ring of the device rests on the limbus of the eye, where the white meets the colored iris at the edge of the cornea. The suction ring and device has three models for different shapes and sizes of faces and eyes. The first model is an arcuate unit with a circular ring with a post that the handpiece fits over and an arc track with a ball bearing guide. The second model is a straight unit or longitudinal design with two parallel tracks with ball bearing cars. The third model is a rotary unit, similar to the arcuate unit but with a horizontal handpiece and a clear applanation lens that flattens the entire cornea and is stationary. The suction ring has an extension or “pipe” for attaching a suction pump. A disposable plastic hose is used to connect the suction ring to the control console. Multiple suction openings are built into the ring. The ring is machined stainless steel.

The handpiece of the cutter is composed of a wire or band that does the cutting of the epithelial layer and an ultrasound generator coupling. For the arcuate model suction ring the handpiece fits over the post with a bow or extension to the ball bearing car and track. There is a sleeve on the side of the cutter to slip over the post of the suction ring. The straight and the arcuate handpiece has a roller positioned in front of the cutter to flatten or applanate the cornea. The roller is moveable but may be a simple bar used to flatten or applanate the cornea immediately before the cutter engages the epithelium. Within the handpiece is a retraction spring for the wire or band cutter. Preferably, the only control on the handpiece is a small button that acts as a trigger to retract the cutter. The cutting head is positioned on a post of the suction ring or fit on top of the ring into a ball bearing track.

In surgery, the rotary handpiece pivots on the post of the arcuate ring but is in a horizontal configuration rather than vertical. The wire or band cutter is advanced across the cornea to a preset stop to create a hinge of epithelium. The rotary unit applanates the cornea by means of a stationary clear plate or lens. A retractable blade is passed beneath the epithelium, with the applanation plate pressing and holding the epithelium in place. The ultrasound is turned on during this movement to aid in the fashioning of the epithelial flap. The cutter (cutting element) is connected to the ultrasound so that the cutter will vibrate at a high frequency during the lift of the epithelium. A vacuum line may be attached to the plate to further stabilize the epithelium. At the end of the pass of the blade, the blade is retracted by pushing the trigger so that the entire device can be lifted off the eye without any further manipulation to the epithelium. Any of the three models of the device could accommodate a motorized drive if desired. The epithelial flap can then be reflected back at the surgeon's leisure to expose the corneal surface for ablation. After the ablation, the epithelium would be repositioned and would adhere to the corneal surface. A standard ultrasound control wire is used to connect the handpiece to the control console.

The ultrasound is connected to an ELF ultrasound control console that controls the power of the pulsations. The suction pump for the vacuum connection to the suction ring is contained in the same console. A reserve vacuum pump is fitted to the console in case of loss of suction.

There are several unique aspects to this device. The first unique feature is the thin band or wire used as a cutter. Much like a wire used to cut food, the wire or band would pass under the epithelium, separating it from Bowman's with the least possible trauma. The epithelium would be laid back on Bowman's immediately after passage of the wire unlike current keratomes that feed the flap up into the keratome where it has to be unfed on the reverse pass of the keratome. In the ELF device there would be no need for a back pass. The second unique aspect is the retracting wire/band cutter. At the end of the forward pass in a standard keratome, the flap has been fed into the device so that the keratome must reverse and play the flap out before the keratome can be removed from the eye. Otherwise, the flap would tear as the keratome is lifted off the cornea. The retracting cutter of the ELF device would retract into the handpiece after the forward cut leaving the flap in place on the cornea so that the handpiece could be removed without a reverse pass. The use of the stationary applanation lens rather than a moving one is unique and will further lessen manipulation of the epithelium. This stationary applanation lens may be fitted to any of the three models of the ELC device. Another unique feature is the ultrasound. All other keratomes rely on oscillating or rotating blades driven by electrical motors to make the cut. As the flap typically lies on the blade during the reverse pass, an oscillating blade would stress and tear a thin fragile epithelial flap. The ELF device cutter would vibrate at a very high frequency (40,000) to ease the dissection of the epithelium from Bowman's. The cutter may be used at other very high frequencies sufficient to effectuate cutting of the corneal tissue. The ball bearing guide has not been used in keratomes before. Most keratomes rely on electrical motors to push them across the eye. As the ELF handpiece would be vibrating, guided by a ball bearing track, and only required to lift off the thin epithelium, a motor drive is not required.

The ELF technique would combine the advantages of LASIK and PRK. As in LASIK the inner cornea would be covered at the end of the surgery for patient comfort and protection against infection. As in PRK more cornea would be available for ablation with the 60-micron epithelial flap as compared to the 160-micron stroma and epithelial flap of LASIK. Further, as in PRK, there would be less biomechanical effect on the eye from a thick flap and fewer flap complications.

Although the ELF device cutter preferably is used to dissect the epithelium, the ultrasonically vibrating blade or wire may also be used to create a resection of a selected thickness. For example, the ELF may be used to cut a flap below Bowman's and deeper into the corneal stroma.

Moreover, the microkeratome known in the art today may be adapted to utilize the ultrasonic blade or ultrasonic wire in place of the fixed or oscillating blade of today's microkeratomes.

Moreover, the embodiments described are further intended to explain the best modes for practicing the invention, and to enable others skilled in the art to utilize the invention in such, or other, embodiments and with various modifications required by the particular applications or uses of the present invention. It is intended that the appending claims be construed to included alternative embodiments to the extent that it is permitted by the prior art.

I claim:

1. An ultrasonic microkeratome device, comprising:
a cutting head assembly comprising an ultrasonic cutting element suitable for corneal resections.

2. The ultrasonic microkeratome of claim 1, wherein the cutting element is a blade.

3. The ultrasonic microkeratome of claim 1, wherein the cutting element is a wire.

4. The ultrasonic microkeratome of claim 1, wherein the cutting element is retractable.

5. The ultrasonic microkeratome of claim 1, wherein the cutting element is removable.

6. The ultrasonic microkeratome of claim 1, further comprising a guide assembly for placement on the ocular globe.

7. The ultrasonic microkeratome of claim 6, further comprising a support assembly connected to said guide assembly and supporting said cutting head assembly for movement of the cutting element along a cutting path of the cornea of the ocular globe.

8. The ultrasonic microkeratome of claim 1, wherein the cutting element vibrates at a very high frequency.

9. The ultrasonic microkeratome of claim 1, wherein the cutting element vibrates at very high frequencies sufficient to effectuate cutting of the corneal tissue.

10. The ultrasonic microkeratome of claim 1, further comprising a drive assembly for driving said cutting head assembly such that said ultrasonic cutting element travels along a predetermined path.

11. The microkeratome device of claim 10, wherein the predetermined path is a generally arcuate or longitudinal path.

12. An ultrasonic microkeratome system, comprising:
a means for retaining and positioning the eye on which surgery is to be performed;
a cutting head assembly including an ultrasonic cutting element; and
and a coupling member for detachably coupling the retaining and positioning means and cutting head assembly while permitting movement of the cutting head assembly relative to the retaining and positioning means along a generally arcuate or longitudinal path.

13. The ultrasonic microkeratome system of claim 12, wherein the cutting element is a blade.

14. The ultrasonic microkeratome of claim 12, wherein the cutting element is a wire.

15. The ultrasonic microkeratome system of claim 12, wherein the cutting element is retractable.

16. The ultrasonic microkeratome system of claim 12, wherein the cutting element is removable.

17. The ultrasonic microkeratome system of claim 12, wherein the cutting element vibrates at a very high frequency.

18. The ultrasonic microkeratome system of claim 12, wherein the cutting element vibrates at very high frequencies sufficient to effectuate cutting of the corneal tissue.

19. The ultrasonic microkeratome system of claim 12, wherein the retaining and positioning means comprises a guide means extending in a generally arcuate or straight path.

20. A microkeratome device for use with an eye, said device comprising;
a base assembly including a suction ring for attachment to said eye;
a cutting head assembly including an ultrasonic cutting element; and
a drive assembly for driving said cutting head assembly such that said ultrasonic cutting element travels along a predetermined path.

21. The microkeratome device of claim 20, wherein the predetermined path is a generally arcuate or longitudinal path.

22. The microkeratome device of claim 20, wherein the cutting element is a blade.

23. The microkeratome device of claim 20, wherein the cutting element is a wire.

24. The microkeratome device of claim 20, wherein the cutting element is retractable.

25. The microkeratome device of claim 20, wherein the cutting element is removable.

26. The microkeratome device of claim 20, wherein the cutting element vibrates at a very high frequency.

27. The microkeratome device of claim 20, wherein the cutting element vibrates at very high frequencies sufficient to effectuate cutting of the corneal tissue.

28. The microkeratome device of claim 20, wherein the suction ring is an arcuate unit.

29 The microkeratome device of claim 20, wherein the suction ring is a straight unit.

30. The microkeratome device of claim 20, wherein the suction ring is a rotary unit.
